(19) United States
(12) Patent Application Publication
(10) Pub. No.: US 2013/0102859 A1
(43) Pub. Date: Apr. 25, 2013

(54) SYSTEM AND METHOD FOR DETECTING SYMPTOMS OF HYPOGLYCEMIA

(71) Applicant: Amir Schechter, Kadima (IL)

(72) Inventor: Amir Schechter, Kadima (IL)

(73) Assignee: GILI MEDICAL LTD., Migdal Haemek (IL)

(21) Appl. No.: 13/805,815

(22) PCT Filed: Jun. 20, 2011
(86) PCT No.: PCT/IL11/00488

§ 371 Date: Dec. 20, 2012

Related U.S. Application Data
(60) Provisional application No. 61/344,273, filed on Jun. 22, 2010.

(51) Int. Cl. A61B 5/0205; A61B 5/02; A61B 5/00; A61B 5/11

(52) U.S. Cl. CPC A61B 5/0205; A61B 5/1118; A61B 5/02007; A61B 5/746; A61B 5/6802; A61B 5/681; A61B 5/7246; A61B 5/7282
USPC 600/301; 600/483

(57) ABSTRACT

An improved system and method for detecting a hypoglycemic event of a diabetic individual is provided. The system of the invention includes at least three sensors for monitoring the respective physiological parameter selected from the following list: heart rate, motorial activity, respiring rate, vasoconstriction, temperature of the skin and galvanic resistance of the skin of the user. The system automatically alerts the user and/or a member of medical care personnel in a case that at least three different symptoms of a hypoglycemic event, each of which associated with a respective physiological parameter, simultaneously occur. According to the method of the present invention basal levels and basal rates of changes of the respective physiological parameters are generated and repeatedly updated. These basal levels and/or rates provides for detecting changes that go beyond predefined limits for determining a symptom of a hypoglycemic event which is associated with the respective physiological parameter.

### FIELD OF THE INVENTION

The present invention generally relates to the field of physiological measurement. More particularly, the present invention relates to a system and method for monitoring motorial activity and/or tremor, heart rate, respiration rate, peripheral vasoconstriction, surface temperature and electrical conductivity of the skin as means for detecting symptoms associated with hypoglycemic event of a diabetic individual.

### BACKGROUND OF THE INVENTION

Hypoglycemic events are typically associated with a range of symptoms such as dizziness, impaired coordination, mental confusion, and altered behavior, increased and irregular heart rate, enhanced perspiration, tremor and heavy respiration. An untreated hypoglycemic event may lead to extreme hypoglycemia that may result in spasms, coma, brain damage or even death. Therefore the development of noninvasive methods and systems for detecting symptoms of hypoglycemic events are ongoing.

A few exemplary systems and methods for detecting some of the above mentioned hypoglycemic symptoms that provide for alerting a user of an occurring hypoglycemic event are listed below. A system for detecting the level of perspiration is disclosed in U.S. Pat. No. 4,365,637. A system incorporating measurement of the skin temperature and the level of perspiration is disclosed in U.S. Pat. No. 4,509,531. However, a study in which the last system was used along 1444 nights by insulin dependant diabetic subjects reveals a significantly high rate of false alarms. The main cause for the high false alarm rate is interpreted by enhanced sleep sweatiness that is not caused by hypoglycemic event, as is reported in Diabetes Care, Vol. 6, November 1983.

A system and method that is capable of screening out cases in which the skin temperature of a diabetes subject decreases and/or cases in which his or her perspiration level increases due to environmental situations other than hypoglycemic events is disclosed in U.S. Pat. No. 7,052,472. The disclosed system automatically derives the rate of change of the skin temperature as well as the level of perspiration and compares them to respective basal rates that are continuously derived by the system. However, cases in which a hypoglycemic event is not associated with an increased sweatiness cannot be detected by such a system.

A system and method for monitoring heart rate especially including the length in time of the QT intervals by means of ECG signals and the mean or peak frequency of the alpha wave by means of EEG signals in addition to monitoring the impedance of the skin for monitoring perspiration level is disclosed in U.S. Pat. No. 7,450,986. The disclosed method seems more robust and less susceptible to false alarms. However such system requires attaching electrodes to the skull of the user in addition to attaching electrodes to his or her chest. Such setup is somewhat cumbersome and less convenient compared to wristwatch like systems that can be attached to a limb of the user.

Another system that monitors the level of perspiration, detects tremor and looks for an increase in the heart rate is disclosed in Japanese patent application the publication number of which is JP20082535660A2.

A system and method for monitoring hemodynamic parameters, such as a radius of a peripheral blood vessel, by means of light scattering measurements is disclosed in U.S. Pat. No. 6,280,390.

Therefore a system capable for detecting hypoglycemic events which is characterized with high sensitivity and low false alarm rate is called for.

### BRIEF DESCRIPTION OF THE DRAWINGS

FIG. 1 is an exemplary time profile of motorial activity of a type I diabetic subject;

FIG. 2 is an exemplary time profile of heart rate of the same individual whose motorial activity is shown in FIG. 1;

FIG. 3 is an exemplary time profile of skin temperature of the same individual whose motorial activity is shown in FIG. 1;

FIG. 4 is an exemplary time profile of galvanic resistance of the skin of the same individual whose motorial activity is shown in FIG. 1;

FIG. 5 is an exemplary time profile of glucose suspended in the blood of the same individual whose motorial activity is shown in FIG. 1;

### DETAILED DESCRIPTION OF THE PRESENT INVENTION

In accordance with the present invention an improved non-invasive system and method for detecting a hypoglycemic event is provided. The method of the present invention provides for significantly reducing the rate of false alarms and yet retaining a high sensitivity of detecting hypoglycemic events at their early stages. A system of the invention is adapted and arranged for monitoring the following physiological parameters: i. heart rate; ii. motorial level; iii. vasoconstriction and skin temperature, and iv. galvanic resistance of the skin. According to the method of the present invention changes in the level of each of these physiological parameters are recorded and compared to respective scales. A symptom of a hypoglycemic event is determined in any of the following cases: a. an increase in the heart rate which crosses and exceeds a respective predefined threshold; b. an increase in the motorial level that crosses or exceeds a respective predefined threshold; c. a decrease in the galvanic resistance of the skin that down crosses a respective predefined galvanic resistance level, and d. vasoconstriction and/or a decrease in the temperature of the skin which exceeds a respective predefined rate of cooling. A case in which any of the symptom of a hypoglycemic event is determined despite the fact that a hypoglycemic event has not occurred is considered as a false alarm. Reducing the rate of false alarms according to the method of the present invention is accomplished by diagnosing a hypoglycemic event when at least three different symptoms are simultaneously determined. However in this case the conditions for determining a symptom are somewhat less stringent. Namely, instead of independently comparing the magnitude of the change in the level of each physiological parameter to a respective threshold a combination of such deviations is first evaluated and its value is compared to a different predefined threshold, as further described infra.

Systems of the invention further monitor the ambient temperature of a user. In cases in which the ambient temperature changes, the level by which it is changed is considered for reducing biases that might be added to the measured temperature of the skin. A system of the invention further monitors the galvanic resistance of the skin for deriving the level of perspiration. Relatively high ambient temperature may impact the level of perspiration thereby add biases to, and/or obscure, the measured galvanic resistance of the skin. Therefore the measured levels of the galvanic resistance are such weighted according to the method of the present invention to reduce their impact in diagnosing hypoglycemic events in cases in which the ambient temperature exceeds a predefined temperature level. A three axes accelerometer incorporated in a system of the invention provides for monitoring the level of movements of the user and for detecting tremor.

An exemplary sensor for noninvasively monitoring vasoconstriction and evaluating the magnitude of the radii of the blood vessel considered has two light detectors that are spaced apart by a predefined distance such positioned that their active faces face the skin of a user. Each light detector comprises an illuminating LED that is operative in the range of near infra-red (NIR) and a photodiode of the same spectral range for receiving the light reflected off the skin. These light detectors are such placed onto the skin of a user that each light detector faces a segment of a peripheral blood vessel or a set of blood capillaries. Measuring the radius of a peripheral blood vessel is accomplished, such as described in U.S. Pat. No. 6,280,390. However, detecting the phenomena of vasoconstriction can be accomplished by means of only one such light detector. Namely monitoring vasoconstriction is accomplished according to the method of the present invention by measuring the intensity of infrared light reflected off the skin by means of one such light detector. A case in which the magnitude of the radius decreases such that the amplitude of the reflected light decreases below a given predefined level is considered according to the method of the present invention as a symptom of suspected hypoglycemic event. One such light detector can implement according to the present invention as a multipurpose sensor for monitoring pulse or heart rates as known and for monitoring vasoconstriction as well as for implementing a non-contact temperature sensor for measuring the temperature of the skin. Obviously the amplitude of the intensity of light scattered off the skin and received by the photodiode considered is proportional to the quantity of blood that passes through the peripheral blood vessels. The rate in which peaks of the time profile of this amplitude occur is equivalent to the pulse rate. Additionally, the intensity of the reflected light averaged along a time interval that include a few pulses, is proportional to the average quantity of blood that passes through the peripheral blood vessels along a time unit and therefore functionally dependent (when a single valued function is considered) on the temperature of the skin. Therefore by employing a suitable calibrating curve the respective temperature of the skin is derived. Whenever the same system component is used as a multipurpose sensor it is regarded according to the present invention as a bunch including the respective number of different sensors that are independently incorporated into the system of the invention.

An improved and non-invasive system for detecting hypoglycemic events according to a preferred embodiment of the present invention has two separate units. One of the units, which is the first unit, is structured such as a wristwatch having a chassis to which a strap attachable to a limb of a user is connected. This unit is equipped with two non-contact temperature sensors. A light detector such as described above implements one of these non-contact temperature sensors. This light detector is such positioned at the inner surface of the chassis that it faces a peripheral blood vessel or a bunch of capillaries. The same light detector is regarded as a second sensor that additionally provides for measuring the heart rate, as described above. The other temperature sensor is such connected to the external surface of the chassis that it cannot face the skin of the user. Alternatively according to another preferred embodiment of the present invention a cable connects the second temperature sensor to this unit such that it can be placed and directed aside of the user. A sensor for monitoring the level of perspiration is implemented by means of two spaced apart electrodes (by a predefined distance). Both electrode are attached to the inner surface of the strap such that a segment of each electrode touches the skin of the user. Both electrodes are electrically connected to a an electric circuitry for measuring the galvanic resistance a segment of the skin which electrically connects between them. This electric circuit is attached to the chassis. A three axes accelerometer connected to a dedicated electric circuitry implements the movement, or rather the motorial activity sensor which is also housed in the chassis of the first unit. All the above mentioned sensors are connected to respective sampling and digitizing units which are further connected to a programmable processor having a memory. This processor inoperative inter alia in comparing the monitored levels of the physiological parameters considered to respective scales stored in its memory; carrying out numerical calculations such as for deriving the heart rate, or the spectrum of the motorial activity of the user. The processor is similarly installed in the chassis of the first unit. Electrical cable that includes wires respectively leading control signals and power connects between the first and second units of the system. The second unit is housed in a case that can be placed near by the user. The second unit includes electrical power supply and an alarm member implemented by a buzzer which is activated by the processor of the first unit. Additional electrical cable provides for connecting the power supply to an external power line. Alternatively, the power supply is substituted with rechargeable batteries. Optionally the second unit is further electrically connected by means of a wireless link, such as by employing Bluetooth protocol to a remote alarm unit for alerting a care providing personnel member, or a relative of the user, in cases of diagnosed hypoglycemic events.

Monitoring any of the aforementioned physiological parameters provides for deriving a respective instantaneous levels as well as deriving the respective basal levels the monitored parameters and/or rates of their respective changes. Deriving the instantaneous level and/or rate of change of a monitored parameter is accomplished as known by averaging a predefined number of samples. Averaging can be accomplished by means of a moving window technique which considers a relatively small number of elements; and/or averaging by means of a FIFO filter that considers samples received along a relatively small time intervals which are referred hereinafter as measuring time intervals. These measuring time intervals are respectively of a few seconds up to a number of minutes. The respective basal levels and/or rate of changes are derived according to the present invention by averaging measured instantaneous levels along a relatively long time interval, which is referred hereinafter as the basal time interval and ranges along a few dozens of lengths of measuring intervals, such as of a number of dozens of minutes. In accordance with the method of the present invention statistical analysis is continuously carried out by which characteristic statistics, and statistical moments such as mean value and standard deviation of each of the physiological parameters are repeatedly updated. Such updating goes on along each and every time in which the system of the invention is used. Therefore, at initial stages of each daily operation long term mean values can be used instead of respective basal levels as long as the respective basal level has not stabilized in time yet. The currently updated basal levels, and characteristic statistics, as well as the long term statistics are stored in the memory of the system processor to be used as respective scales against which the instantaneous levels are compared. Additionally, in accordance with another embodiment of the present invention, whenever an instantaneous level of any of the physiological parameters exceeds the basal level by more than a predefined portion of the magnitude of respective long term standard deviation, a hypoglycemic event is diagnosed regardless of the measured levels of the other physiological parameters.

The instantaneous level of the temperature of the skin and/or the ambient temperatures is repeatedly measured at a predefined repetition rate (which equals the reciprocal of the length of the measuring time interval). In cases in which the currently measured instantaneous skin temperature decreases compared to the respective basal level at a rate faster than the rate by which the ambient temperature decreases and/or at a rate that exceeds a predefined threshold, such temperature decrease is considered according to an embodiment of the method of the present invention for determining a symptom of decreasing skin temperature that is indicative to a hypoglycemic event.

The instantaneous level of the galvanic resistance of the skin is compared to the respective scale such as typically recorded in handbooks. Alternatively such a scale is experimentally derived by carrying out suitable statistical analysis for determining typical galvanic resistance levels that respectively correspond to the resistance of a skin under different levels of sweatiness. When the currently measured resistance gets down compared to such scale and crosses a predefined threshold a symptom of hypoglycemic event associated with perspiring is determined. When the ambient temperature is lower than a predefined temperature it is unlikely that such perspiring is caused by the ambient temperature. Therefore such crossing of the threshold of galvanic resistance is considered as a sweatiness symptom of suspected hypoglycemic event.

Signals of the three axes accelerometer are repeatedly sampled along each axis at sampling frequencies which range a number of dozens of cycles per second (CPS) up to say ten dozens of CPSs. A spectrum of these signals is derived according to a preferred embodiment of the method of the present invention, as described following. First the number of crossing along any of the three axes of a predefined level of acceleration, which occur along one second is counted in each and every second. Then the system time tags and records those seconds that such crossing of this acceleration level has occurred at least a predefined number of times per second, say a dozen crossing per second. Such seconds are considered according to the present invention as motorial active seconds. Then the number of motorial active seconds that are included within a measuring time interval having a predefined length, say of a given number of minutes, is referred hereinafter as the measured number of active seconds, its value is time tagged and recorded. Values of measured numbers of active seconds are averaged along a basal time interval for deriving the basal number of motorial active seconds that are included within a measuring time interval. The length of a basal time interval equals the length of the aforementioned measuring time interval multiplied by, say, dozen times. A symptom of a hypoglycemic event associated to the increased motorial activity is determined when the number of active seconds included within the current measuring time interval exceeds the basal value by at least a given number. The various thresholds, namely the level of acceleration considered for determining a crossing, the number of crossing above which a second is regarded as motorial active second, as well as the threshold for determining a symptom that whenever the number of active seconds that are included in a measuring time interval exceeds it, are experimentally derived by conducting statistical analysis of measurement during trials in which insulin dependent diabetic subjects take part.

Alternatively a spectrum of the cyclic movements such as of heart rate and/or mechanical vibrations can be computed such as by carrying out DFT calculations. Abrupt movements of the user that are typical to tremor are characterized with predefined frequencies. Similarly respiration rates can be derived from such computed spectra of the amplitude of the pulses measured with the light detector. By subtracting the magnitudes of the relevant frequency levels from the respective basal magnitudes and comparing the derived differences with respective threshold levels provides for detecting tremor as well as for detecting an increase in the respiration rate. Hence a system that has three axes accelerometer and a light detector is capable for detecting symptoms of increased movement, tremor and increased respiring rate in addition to monitoring vasoconstriction and detecting events associated with cooling down of the skin. One light detector in combination with respective software programs installed in the system processor provides according to the present invention instead for measuring levels of four different parameter such as accomplished by means of four independent sensors. Embodiment variants of the system for detecting hypoglycemic event in which such light detector is incorporated and used as multipurpose sensor are in accordance with the present invention. Such multipurpose sensor is respectively regarded hereinafter as a bunch of two, three and/or four different sensors, each of which is independently linked to the system processor.

A basal respiration rate is derived by averaging instantaneous respiring rates. The instantaneous respiring rates are derived in consideration with the lower frequencies of the spectrum of the DFT analysis of the pulses of blood their amplitude is measured such as with a light detector, as known. The level by which an instantaneous level deviates from the respective basal rate is compared to calibrating scale. When this deviation exceeds a predefined threshold a symptom of heavy respiring is determined.

In cases in which all the above mentioned symptoms simultaneously occur a hypoglycemic event is detected according to an embodiment of the method of the present invention. Alternatively scores associated with at least some of the above mentioned parameters are evaluated and their summation exceeds a respective predefined threshold level a hypoglycemic event is diagnosed. Obviously in cases that smaller number of symptoms is simultaneously determined the rate of false alarm increases if one retains the same sensitivity of detection.

Following is a description of the process for diagnosing a hypoglycemic event according to a preferred embodiment of the present invention. This process is applicative in cases in which at least three physiological parameter are monitored, wherein those three are selected out of the following physiological parameters (i) motorial activity, (ii) heart rate, (iii) respiring rate, (iv) temperature of the skin or preferably vasoconstriction without evaluating the radii of the blood vessels, and (v) galvanic resistance of the skin.

First, levels of the differences between the instantaneous levels measured at each and every measuring cycle and the basal level of the respective monitored physiological parameters (except for the perspiration sensor) are scored. Scoring is accomplished by mapping the respective maximal level of any of the differences according to the method of the present invention to one. (similarly any instantaneous level measured for the physiological parameter considered, that is higher than such maximal level, is also mapped to one.) any level of such difference which equals zero or is negative is mapped to zero. The full range between the maximal level of difference is linearly mapped into the open interval (0,1). Scoring levels of galvanic resistance of the skin is accomplished by mapping the range of the respective levels of galvanic resistances into the closed interval [0,1] as follows: any resistance which equals or exceeds a predefined maximal level of resistance, such as 250 KO, is mapped to zero. Any resistance which equals or is lower compared to a predefined minimal resistance, say 100 KΩ, is mapped to one. All the levels between these two limits are linearly mapped into the open interval (0,1) in accordance with the equation S=1−(R−100)/150, where S is the score received for these predefined maximal and minimal levels of resistance; R is the level of the measured galvanic resistance given in KΩ. The minimal and maximal levels are derived by statistically averaging measurements results of experiments in which a large number of diabetic participants toke part. The differences that are associated to the physiological parameters other than the levels of galvanic resistance of the skin are scored as follows: first a range of the differences between an instantaneous level and the basal level is selected then the upper end of this range is mapped to one. And differences that are negative or equals zero are mapped to zero. Any intermediate level is respectively scored by dividing its value by the magnitude of the upper limit. Therefore all the intermediate levels are mapped into the open interval (0,1).

Such derived scores for all the monitored parameters are summed together into a combined score. The combined score is further compared to a respective threshold level. When the combined score exceeds a predefined threshold level a hypoglycemic event is detected. In cases of ambient temperature that is too high that might causes the user to perspire, it is preferable to avoid using the perspiration sensor and the threshold for the combined score is lowered accordingly. Such approach provides for retaining the level of sensitivity however on the other hand, the rate of false alarms is somewhat increased. In cases in which the ambient temperature decreases at a rate higher than a predefined cooling rate, it is preferable according to the method of the present invention to avoid using the skin temperature sensor for a while and wait until the ambient temperature stabilizes. A different predefined threshold level for the combined score that does not consider the score associated to skin temperature is used in such case. Such approach provides for retaining the sensitivity level of the system however the rate of false alarm is somewhat increased.

### Example 1

An experiment in which a group of insulin dependent diabetic members, each using a system for detecting hypoglycemic events according to an embodiment of the present invention, is hereby described with reference to

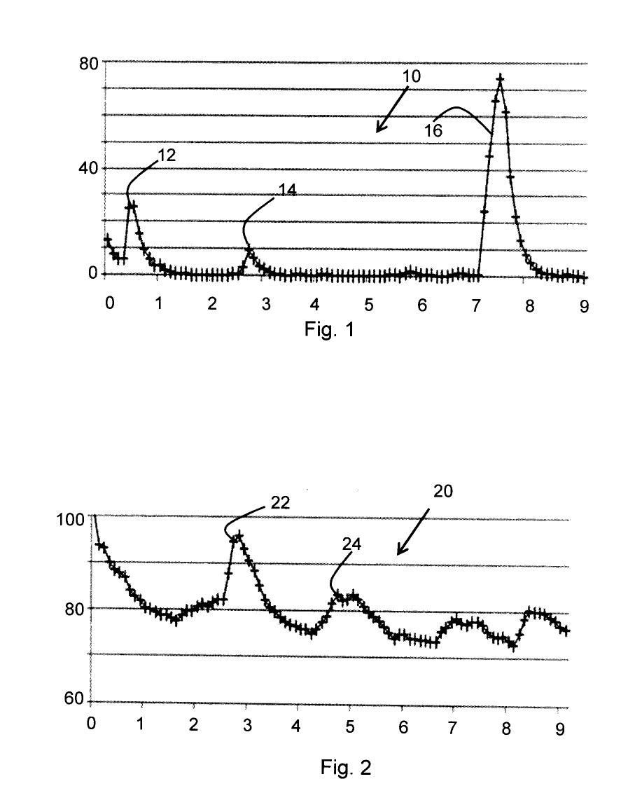

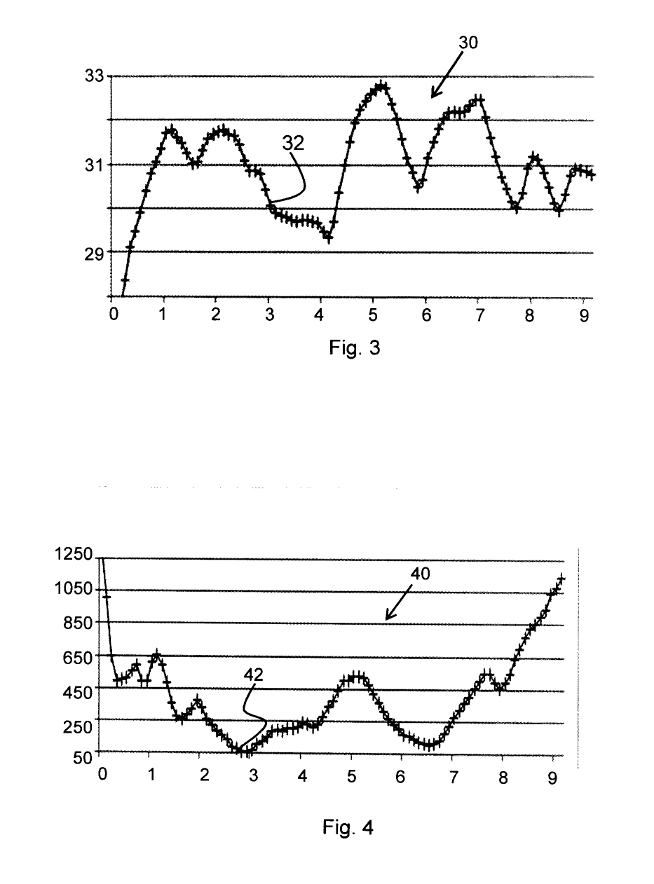

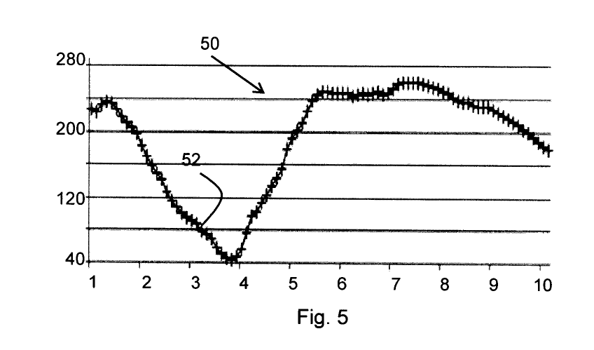

FIGS. 1-5. Time profiles of the motorial activity, heart rate, temperature of the skin, galvanic resistance of the skin, and the level of glucose suspended in the blood, all of which were derived for an exemplary user, are respectively shown in FIGS. 1-5. The horizontal axis shown in each of this graphs indicate time measured in hours starting at the beginning of a night sleep. Identical lengths of the measuring time interval are employed in any of the graphs shown, all of which equals five minutes. The systems employed are structured such as a wristwatch to be worn on an arm of the user. The systems include: a light detector for measuring heart rate; thermistor that implements temperature sensor for measuring skin temperatures; a sensor for measuring galvanic resistance of the skin and three axes accelerometer operated in a mode for measuring motorial activity as described hereinabove. Commercial Glucometer of the type Dexcom Seven Plus continuously intrusively monitored the actual level of glucose suspended in the blood of the users. The scale of the vertical axis of FIG. 1 is dimensionless. Plot 10 is the time profile of the number of motorial active seconds included in any measuring time interval. Peaks 12, 14, 16 can be interpreted as presenting determined symptoms. Namely cases in which a significant change of the number of active seconds currently occurred compared to the basal count. The value of basal level that provide for deriving Peak 12, might be questionable due to stabilization time that could be longer compared to the time elapsed from the moment in which measuring is started. Nevertheless point 14 indicates a symptom of a hypoglycemic event related to motorial activity which simultaneously occurs with symptom of the other three physiological parameters at the same point in time. (This point in time is fifteen minutes prior to three.) Plot 20 presents the time profile of the pulse rate, the units of the vertical axis are beats per minute, measuring the pulse rate is repeatedly accomplished each and every measuring time interval. The peak located close to point 22 corresponds to a symptom of an increased heart rate, by indicating a change in the currently measured pulse rate compared to the basal rate of about nine pulses per minute. Indeed point 22 indicates determining a symptom of a hypoglycemic event related to increased pulse rate, which occurs synchronously with the symptom indicated by point 14. Successive points leading to point 24 of the following peak correspond to changes of three beats per minute at most. Plot 30 presents the time profile of the temperature of the skin, measured in degrees Celsius. Point 32 indicates a state in which symptom of a hypoglycemic event related to falling temperature of the skin is determined. There are additional points along this plot that exhibit decreasing temperature however none of these additional points comply with respective symptoms of motorial activity nor of heart rate. Therefore according to the method of the present invention any of these additional points cannot provide for diagnosing a hypoglycemic event even in cases that there are respective points that belong to the time profile of the galvanic resistance of the skin which respectively present determined symptoms of enhanced perspiring. The scale of FIG. 4 is measured by kilo-Ohms; plot 40 presents the time profile of the galvanic resistance of the skin. Point 42 indicates a point in time in which the combined scoring according to the preferred embodiment of the method of the present invention for diagnosing a hypoglycemic event described hereinabove. Plots 30 and 40 are significantly more oscillatory compared to plots 10, 20 and 50. Indeed the temperature of the skin normally varies along time during the night by a few degrees Celsius. Covering with a blanket and/or exposing the temperature sensor and the organ onto which this sensor is attached to the ambient air, may easily cause such variations. Therefore it is preferable according to the method of the present invention to monitor respiring rates rather than the temperature of the skin, thereby promoting the chances for retaining high sensitivity and a relatively lower false alarm rate.

Furthermore, perspiring can occur independently of a hypoglycemic event. However, most of the hypoglycemic events are associated with heavily perspiring. Therefore lack of perspiring may require more stringent conditions for diagnosing a hypoglycemic event and retaining a permissible rate of false alarms.

Plot 50 present the actual time profile of the level of glucose suspended in the blood of this very user. Point 52 is the very point of this plot that corresponds in time to points 14, 22, 32, 42. Indeed a level of 65 mg/ml of glucose corresponds to a situation of a user who experiences for quite a significant time a hypoglycemic event. (However for the sake of full disclosure a statement should be made with regard to poor calibration of the continuous glucometer at relatively low levels of concentration of glucose, namely levels which correspond to hypoglycemic event can be erroneous by dozens of percents or more since calibrating at such levels is practically impossible).

1-11. (canceled)

12. A method for non-invasively detecting a hypoglycemic event, the method comprising:
detecting a level of heart rate, motorial activity, peripheral vasoconstriction, and skin resistance;
comparing the detected levels of each of the heart rate, motorial activity, peripheral vasoconstriction, and skin resistance to a respective scale and assigning a respective score to each of the detected heart rate, motorial activity, peripheral vasoconstriction, and skin resistance; and
determining a hypoglycemic event when a combined score calculated from the respective scores reaches a predefined value.

13. The method of claim 12, wherein when a hypoglycemic event is determined an alarm is triggered.

14. The method of claim 12, wherein the respective scale of each of the heart rate, motorial activity, peripheral vasoconstriction, and skin resistance is predefined or experimentally derived.

15. The method of claim 12, wherein the score of the skin resistance is normalized to changes in ambient temperature.

16. The method of claim 12, wherein detecting a level of heart rate, motorial activity, peripheral vasoconstriction, and skin resistance is performed by three sensors.

17. The method of claim 16, wherein the three sensors comprise: a skin resistance sensor, an accelerometer, and a light detector.

18. The method of claim 17, wherein the light detector is configured to detect heart rate, peripheral vasoconstriction or both.

19. The method of claim 16, wherein the three sensors are incorporated into a single wearable unit.

20. The method of claim 19, wherein the single wearable unit is a wristwatch.

21. The method of claim 12, wherein detecting a level of heart rate, motorial activity, vasoconstriction, and skin resistance is performed by two sensors.

22. The method of claim 21, wherein the two sensors comprise: a skin resistance sensor and a light detector.

23. The method of claim 22, wherein the light detector is configured to detect heart rate, peripheral vasoconstriction, motorial activity or any combination thereof.

24. The method of claim 21, wherein the two sensors are incorporated into a single wearable unit.

25. The method of claim 24, wherein the single wearable unit is a wristwatch.

26. A system for non-invasively detecting a hypoglycemic event, said system comprising:
at least two sensors configured to detect levels of heart rate, motorial activity, peripheral vasoconstriction, and skin resistance;
a processor configured to:
compare each of the detected levels of the heart rate, motorial activity, peripheral vasoconstriction, and skin resistance with respective scales,
assign a respective score to each of the heart rate, motorial activity, peripheral vasoconstriction, and skin resistance; and
determine a hypoglycemic event when a combined score, calculated from the respective scores, reaches a predefined value.

27. The system of claim 26, further comprising an alarm configured to be activated when a hypoglycemic event is determined.

28. The system of claim 26, wherein the scale of each of the heart rate, motorial activity, peripheral vasoconstriction, and skin resistance is predefined or experimentally derived.

29. The system of claim 26, wherein the score of the skin resistance is normalized to changes in ambient temperature.

30. The system of claim 26, wherein said at least two sensors comprise: a skin resistance sensor, an accelerometer and a light detector.

31. The system of claim 30, wherein said light detector is configured to detect heart rate, peripheral vasoconstriction or both.

32. The system of claim 26, wherein said at least two sensors comprise: a skin resistance sensor and a light detector.

33. The system of claim 32, wherein said light detector is configured to detect heart rate, vasoconstriction, motorial activity or any combination thereof.

34. The system of claim 26, wherein said at least two sensors are incorporated into a single wearable unit.

35. The system of claim 34, wherein said single wearable unit is a wristwatch.

36. The system of claim 26, configured to detect said level of heart rate, said motorial activity, said peripheral vasoconstriction, and said skin resistance using only two sensors: a skin resistance sensor and a light detector.
